(19) United States
(12) Patent Application Publication
(10) Pub. No.: US 2023/0372192 A1
(43) Pub. Date: Nov. 23, 2023

(54) MULTI-MODALITY TREATMENT DEVICE

(71) Applicant: BETTERLIFE12 LLC, Tallahassee, FL (US)

(72) Inventors: Nicole EKHOMU, Tallahassee, FL (US); Twan CAPEHART, Tallahassee, FL (US)

(73) Assignee: BETTERLIFE12 LLC, TALLAHASSEE, FL (US)

(21) Appl. No.: 18/198,346

(22) Filed: May 17, 2023

Related U.S. Application Data
(60) Provisional application No. 63/342,860, filed on May 17, 2022.

(51) Int. Cl. A61H 23/02

(52) U.S. Cl. CPC A61H 23/02; A61H 2201/0153; A61H 2201/0242; A61H 2201/0207; A61H 2201/5025; A61H 2201/0285; A61H 2201/0214; A61N 1/0496

(57) ABSTRACT

A handheld treatment device is configured to apply one or more treatment modalities to a user. The treatment device can include a heater, a massager, and an electrical energy generator that applies current to a user's body via EMS or TENS. The treatment device may also include a thermoelectric cooler. A control interface is configured to activate one or more of the heater, the thermoelectric cooler, the massager, and the electrical energy generator. The treatment device may be battery-powered.

### BACKGROUND

The present application describes a handheld nonsurgical treatment device that can be used to alleviate uncomfortable conditions. People, and athletes in particular, may suffer from muscle pain, stiffness, or soreness after a strenuous workout or after an injury has occurred. As a result, there is a need to provide quick and convenient relief to people who have endured minor injuries of this nature. The treatment device disclosed herein is capable of applying a variety of treatment modalities so that user can easily apply any one of, or several of, the treatment modalities to an afflicted area to alleviate discomfort. As such, the user may have several options for quick and convenient self-treatment.

### SUMMARY

A handheld treatment device is configured to apply one or more treatment modalities to a user. The treatment device can include a heater, a massager, and an electrical energy generator that applies current to a user's body via EMS or TENS. The treatment device may also include a thermoelectric cooler. A control interface is configured to activate one or more of the heater, the thermoelectric cooler, the massager, and the electrical energy generator. The treatment device may be battery-powered.

### BRIEF DESCRIPTION OF THE DRAWINGS

FIGS. 1A and 1B illustrate top and bottom view of the treatment device.

FIG. 2 illustrates a block diagram with a high-level overview of certain hardware components of the treatment device.

FIG. 3 illustrates an alternative embodiment of the treatment device that has a curved treatment surface for treating elbows, knees, et cetera.

FIGS. 4A-4D illustrate exemplary GUI screens of a smartphone app used to control the treatment device.

FIG. 5 illustrates the treatment device with pads attached thereto for electrical therapy such as TENS or EMS.

FIG. 6 illustrates a side cross-sectional view of the interior components of the treatment device.

### DETAILED DESCRIPTION OF EXEMPLARY EMBODIMENTS

Exemplary and non-limiting embodiments of the treatment device will now be described.

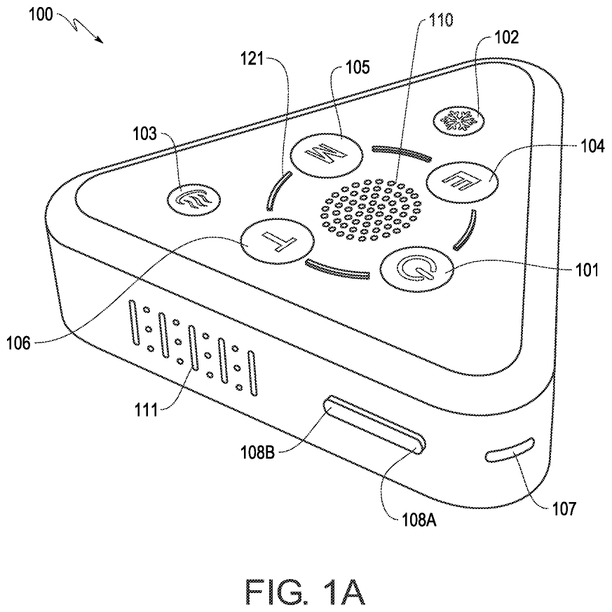

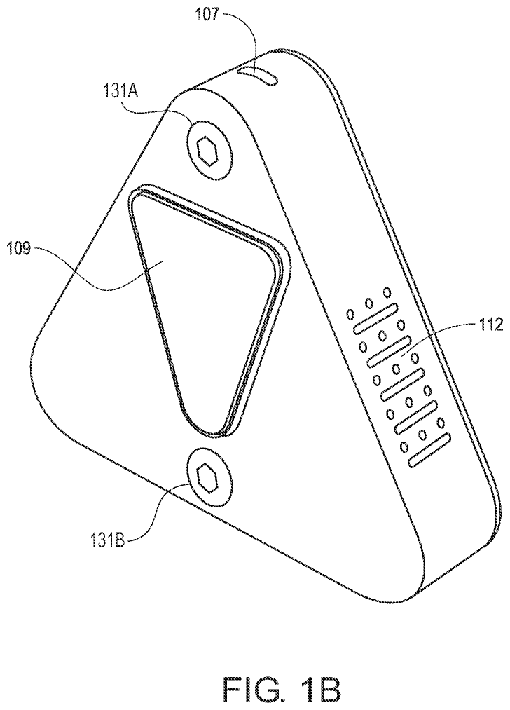

FIG. 1A illustrates a top perspective view of the treatment device 100. The treatment device 100 includes several control buttons. In detail, there is a power button 101 for turning the device on and off. Also included are a cold button 102, a heat button 103, an EMS button 104, a massage button 105, and a TENS button 106.

Each of the buttons 102-106 activates a corresponding function of the treatment device. For example, the cold button 102 causes the treatment device to generate a cooling effect on one surface thereof (this will be described in more detail below). The heat button 103 causes the treatment device to heat up one of its surfaces. The EMS button 104 causes electronic muscle stimulation (EMS) to be applied to a portion of the user's body. The massage button 105 causes the treatment to vibrate in a relatively soothing manner so that a user can apply massage to himself/herself. The TENS button 106 causes transcutaneous electrical nerve stimulation (TENS) to be applied to a portion of the user's body.

The treatment device also includes a data port 107, which may be a micro-USB or mini-USB port. Other connection interfaces could be implemented as well. The data port can enable the transfer and reception of data. For example, the treatment device could be controlled with an external device such as a smartphone, tablet, smartwatch, laptop, or desktop computer via the data port 107. In addition, the treatment device could output data regarding the treatment to the external device, such as the type of treatment applied, a time duration of the treatment, and statistical information related to the treatment such as a temperature applied or current/voltage applied with EMS or TENS.

In another embodiment, the treatment device does not include the data port 107 because wireless communication circuitry is used such as Bluetooth, NFC, or Wi-Fi. The wireless communication circuitry can include an antenna and/or a transceiver that enables Bluetooth, NFC, Wi-Fi, or another communication technique.

Intensity control buttons 108A and 108B are also included. These buttons adjust the intensity of treatment for each of the functions associated with buttons 102-106. For example, a user can adjust the temperature of treatment to be higher or lower with buttons 108A and 108B respectively, and the user can adjust the intensity of the EMS or TENS treatment (adjust voltage, current, frequency, or duration of pulses). The user can adjust the intensity of the massage to be higher or lower in frequency. Note that in FIG. 1A intensity control buttons 108A and 108B are implemented with a single rocker switch, such that opposite ends of the rocker switch when pushed send different signals.

The treatment device also includes a metal plate 109 (FIG. 1B). The metal plate 109 can transmit heat, cooling, and/or vibration to the user's body. For example, when the heating mode is activated, the metal plate 109 becomes warm. When the massage mode is activated, the metal plate vibrates. The metal plate 109 may be a plate of an arbitrary shape, such as a triangle, rectangle, square, or circle. The metal plate 109 could also be curved so that a user can easily apply the treatment to a non-flat body surface, such as the elbow, knee, neck, or foot. That is, the metal plate could have a shape that is complementary to one of the elbow, knee, neck, or foot.

The treatment device may also include magnetic patch connectors 131A, 131B, as shown in FIG. 1B These patch connectors are each configured to connect to a proximal end of an electrode pad so that the treatment device can apply electrical therapy (i.e., EMS or TENS) through the electrode pads. That is, when applying one of these electrical therapies, the battery generates electrical current, the current is converted to an appropriate waveform by electrical conversion circuitry, and the wave form is transmitted through the patch connectors and the electrode pads to the user. Although two patch connectors are shown in FIG. 1B, the number of connectors could be three, four, or a different number. In particular, there may be three patch connectors, each at one vertex of the triangular housing, in another embodiment.

The treatment device 100 also includes vent holes 110, 111, and 112. The vent holes include a small circular shape, and the vent holes 111 and 112 may include an elongated slot shape, or a combination of small circles and elongated slots. The vent holes permit heat to escape from the interior of the treatment device to prevent circuitry and/or the power source from overheating during operation.

The treatment device also includes four semicircular LED lights 121. These lights may all be activated when the power is turned on. In addition, the LED lights change to a different color when a different mode is activated. For example, the lights 121 may be in yellow when TENS is activated. The lights 121 may be green when massage mode is activated. The lights 121 may be purple when EMS is activated. The one light 121 adjacent to the heat button 103 may be yellow, orange, or red when the heating mode is activated. The one light 121 adjacent to the cold button 102 may be blue when the cooling mode is activated.

The housing defining the exterior of the treatment device 100 may be made from plastic, but other durable substances such as metal could conceivably be used. The housing may be formed with two parts, an upper part and a lower part, so that the circuitry and power source can be secured inside the housing prior to the upper and lower parts being secured to each other with screws, adhesive, or the like.

To use the treatment device, the user turns on the device by pressing the power button 101. The power button may need to be pressed for a predetermined amount of time such as three seconds. Then, the user can press one of the buttons 102-106 described above to activate a certain treatment modality. Then the specific treatment mode is initiated, and the user places the treatment device on an area of the body that requires treatment, and the treatment device provides relief to the area. For example, the user could select heat treatment mode, and then place treatment device 100 on his/her wrist if the wrist area is injured and causing pain. Additionally or alternatively, the user could activate massage mode, EMS mode, or TENS mode. Of course, any other area of the body such as the neck, elbows, knees, lower back, and so forth can be treated in the same manner.

Multiple treatment modalities could be activated at the same time. The treatment modalities are grouped in three groups: temperature control, massage, and electrical therapy. One treatment from each group can be activated at the same time. For example, either heat or cold (but not both), massage, and either EMS or TENS (but not both) could be applied at the same time. That is, applying heat and cold at the same time would be counterproductive so the device may be programmed not to apply both of these modalities at the same time. In addition TENS and EMS cannot be applied at the same time due to their distinct requirements. In other words, massage, temperature control, and electrical therapy are independent.

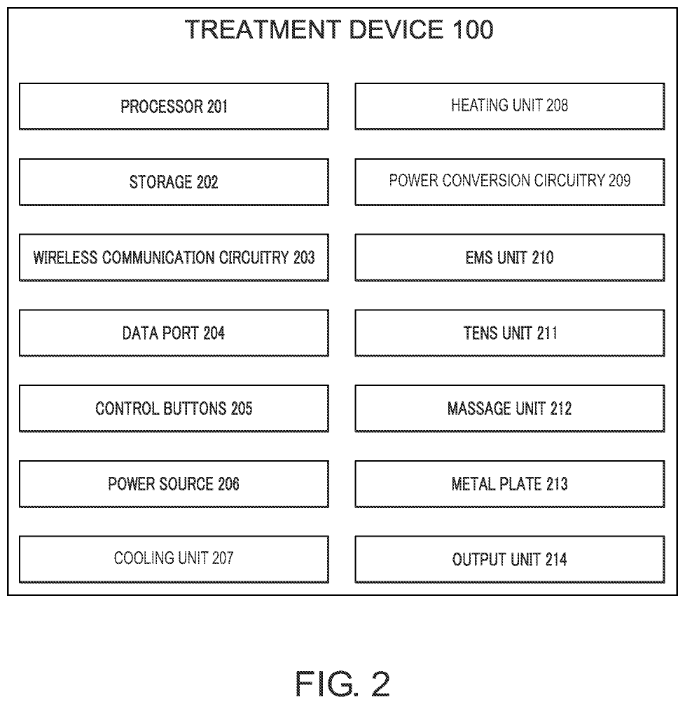

FIG. 2 illustrates an overview of certain hardware components that may be included with the treatment device 100. In practice, some of these components may be omitted under certain circumstances.

The treatment device 100 may include a processor 201 (i.e., a microcontroller, CPU, PLU or the like) that controls overall operations of the device. A RAM and ROM may also be included. The processor 201 can control the other components shown in FIG. 2.

A storage 202 may be included that stores, e.g., software/firmware for controlling operations of the device. The storage 202 can also store data such as a history of treatments applied, statistical information on the treatment, programs for controlling electronic components inside the treatment device, and programs for interfacing with an external electronic device such as a computer, smartphone, tablet, or smartwatch. The storage 202 may be a non-volatile computer readable medium such as flash memory. Other types of non-volatile storage could be used.

Wireless communication circuitry 203 may be included. The wireless communication circuitry 203 may be configured to implement communication via Bluetooth, NFC, Wi-Fi, 4G, 5G, or other communication modalities. The wireless communication circuitry may include an antenna and/or a transceiver. With the wireless communication circuitry, the treatment device 100 can communicate with an external electronic device such as a smartphone, tablet, or smartwatch. For example, a smartphone could be used to control the treatment device.

A data port 204 such as a USB (micro, mini, or USB-A, or USB-C) port may be included. With the data port 204, the treatment device 100 can communicate with an external electronic device such as a smartphone, tablet, or smartwatch. For example, a smartphone could be used to control the treatment device. The data port can also be used to charge the battery from an external power supply, especially when the data port is a standard connection type such as USB A, USB C, USB mini/micro, et cetera.

A plurality of controls buttons 205 may be included. The control buttons are shown in FIG. 1A and described above.

A power source 206 is included. The power source may be a lithium ion battery, but other battery chemistries could potentially be implemented. The power source 206 may also include a charging port where an electrical cable can be inserted to recharge the battery. In an alternative embodiment, the power source could be power conversion circuitry that converts mains power transferred via a plug and wire to electricity suitable for the components inside the treatment device.

The treatment device could even include wireless charging circuitry such as circuitry compliant with the Qi standard and a receiving coil so that the treatment device can be placed on a wireless charging mat to recharge the battery.

The cooling unit 207 generates a cooling effect on a surface of the treatment device. The cooling unit produces the cooling effect via the Peltier effect. With the Peltier effect, electric voltage can be converted to a temperature differential. In detail, electric current is passed through a thermocouple and heat is produced at one side, and a cooling effect is produced at the other side. Thus, by using electrical energy from the battery, a cooling effect can be provided. Multiple coolers may be stacked together to further decrease the temperature of the cooling effect. The cooling unit may also be referred to as a thermoelectric cooler.

The heating unit 208 generates a heating effect on a surface of the treatment device. The heating unit 208 can be implemented by controlling the battery to generate electrical current and have the current flow through a resistive element. The resistive element then increases in temperature, and the high temperature is transmitted to the metal plate. The user can then apply the metal plate portion to the user's own body surface.

The power conversion circuitry 209 may include a boost converter circuit (a DC-DC converter) that steps up voltage output by the battery to a higher voltage suitable for EMS or TENS. The power conversion circuitry 209 may connect to the EMS unit and/or the TENS unit. The power conversion circuitry 209 may include a pair of boost converter circuits, one for each of the EMS unit and the TENS unit. The power conversion circuitry 209 may transmit the boosted electrical signal to a set of electrodes that are placed on the user's skin.

The EMS unit 210 applies electrical muscle stimulation (EMS) to the user of the treatment device. For example, electrical energy from the battery can be converted into electrical signals of an appropriate voltage and current via the power conversion circuitry 209, and applied to the user's skin via electrodes as described above.

The TENS unit 211 applies transcutaneous electrical nerve stimulation (TENS) to the user of the treatment device. For example, electrical energy from the battery can be converted into electrical signals of an appropriate voltage and current via the power conversion circuitry 209, and applied to the user's skin via electrodes.

EMS is a type of therapy that involves applying electrical energy to a user's skin via a pair of electrodes, and the electrical energy is calibrated so as to cause the user's muscles to contract. EMS can thus be used for strengthening of muscles, rehabilitation of muscles, and/or improving local blood circulation. EMS may involve applying electrical stimulation at 10-150 milliamps and 0-150 volts, at a frequency of 1-100 Hertz. When the intensity of the EMS is adjusted via the intensity control buttons, the frequency and the amplitude of the EMS may increase or decrease depending on whether intensity is increased or decreased.

TENS also involves applying electrical energy to a user's skin via at least one electrode or a pair of electrodes, but the effect and purpose is different than EMS. That is, TENS is used for alleviating pain, rather than muscle contraction. TENS applies an electrical current that prevents pain signals from being transmitted to the brain. The TENS therapy may involve applying electrical stimulation at 15-50 milliamps and 25-100 volts in order to provide effective pain relief. The frequency of stimulation may be pulses of 10-100 Hertz. The intensity of the TENS treatment can be changed by adjusting voltage or current, i.e., voltage regulation that selects a set voltage or current selects that maintains a set current. The duration of the pulses may be 50-200 microseconds. The pulse pattern for TENS may be a continuous pattern, a burst pattern with a rapid series of pulses followed by a stoppage period, or a modulated pattern with a series of pulses that increase in amplitude.

In the present case, the treatment device 100 can apply either EMS or TENS depending on the user's selection.

The treatment device may include 2-4 electrode pads that attach to a user's skin, and the electrode pads connect to the main body of the treatment device via wires. The wires are attached to magnetic patch connectors 131A, 131B described above. The electrical energy from TENS or EMS is applied to the user through the electrode pads and wires.

In one embodiment a medicinal substance such as CBD or lidocaine may be disposed on the electrode pads. This medicinal substance can reduce pain that the user is experiencing and provide a more soothing experience.

The massage unit 212 is configured to generate vibration. For example, the massage unit may be implemented with a motor that is unbalanced and drives an object to oscillate at a given frequency, which causes the entire treatment device 100 to vibrate. The treatment device can then be applied to the user's body to provide a massaging effect. When the intensity of the massage is increased or decreased, the speed of the motor can be correspondingly increased or decreased, so that the frequency of the vibration changes.

The metal plate 213 may be a plate of an arbitrary shape (triangle, circle, rectangle, curved plate, et cetera) attached to the treatment device that transfers heat, cooling, or vibration, as described above.

The output unit 214 may be the above-described LED lights, and additionally or alternatively an LCD display screen, or speaker. The output unit 214 could even be formed with all three of these components. The display screen could visually display information such as a power state, a type of treatment being applied, and statistical information on the treatment being applied, such as the temperature, level of intensity, or duration of treatment. Battery life could also be displayed. The speaker could output the same information in audible form. Note that the display screen could be omitted to reduce manufacturing costs. Even the speaker could be omitted in some embodiments. That is, any one from among LED lights, the display screen, or the speaker could form the output unit as well.

The treatment device may include at least one electromyography (EMG) sensor. The EMG sensor measures electrical signals generated by a muscle. The EMG sensor can include or be implemented with two electrode pads that are attached to a user's skin. After the electrode pads are attached, electrical activity that occurs with muscle movement or contraction is then detected. The sensor readings can be sent to the processor for analysis and/or display on a display screen of the user's smartphone.

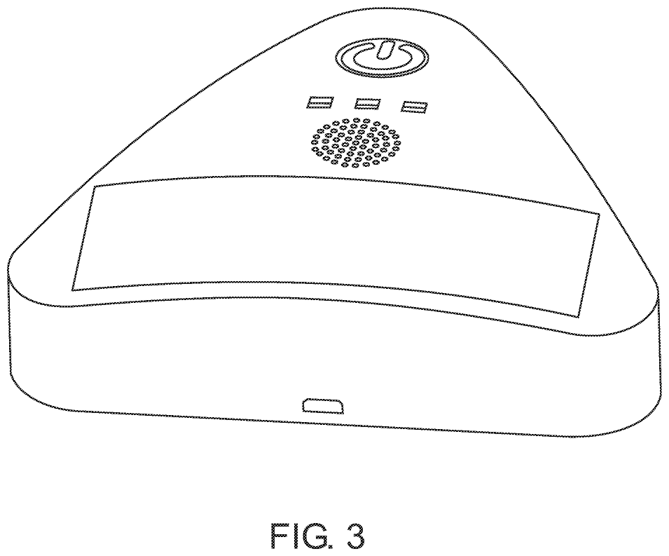

FIG. 3 illustrates another embodiment of the treatment device that has a curved surface on the top side.

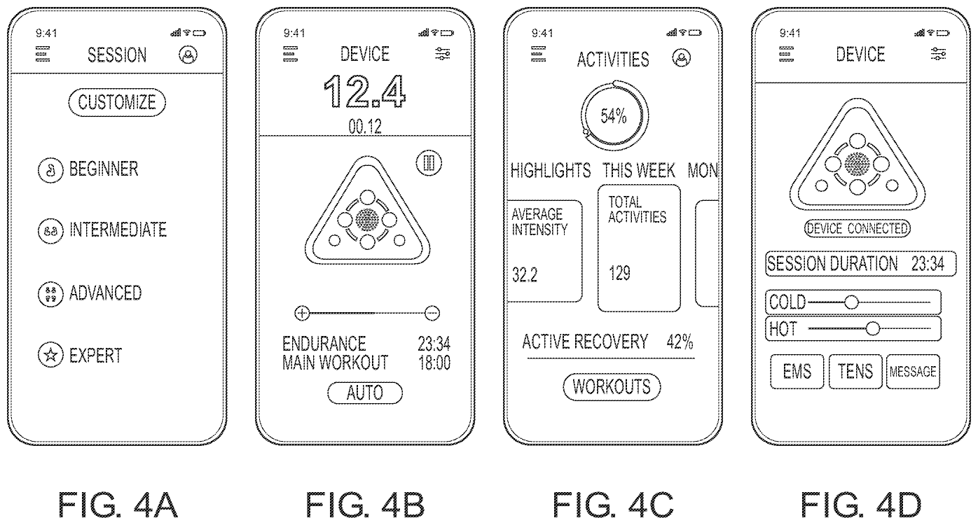

FIG. 4A-4D illustrate exemplary GUI screens that may be displayed on a smartphone, tablet, or other electronic device for controlling the treatment device and statistics regarding the treatment. In FIG. 4A, different modes such as beginner, intermediate, advanced, and expert may be selected. In FIG. 4B, a duration of treatment may be displayed. In FIG. 4C statistics such as average intensity, a completion rate of predefined tasks, and a recovery percentage can be displayed. In FIG. 4D, a session duration, temperature controls, and a treatment device mode such as EMS, TENS, or massage can be displayed and selected by a user.

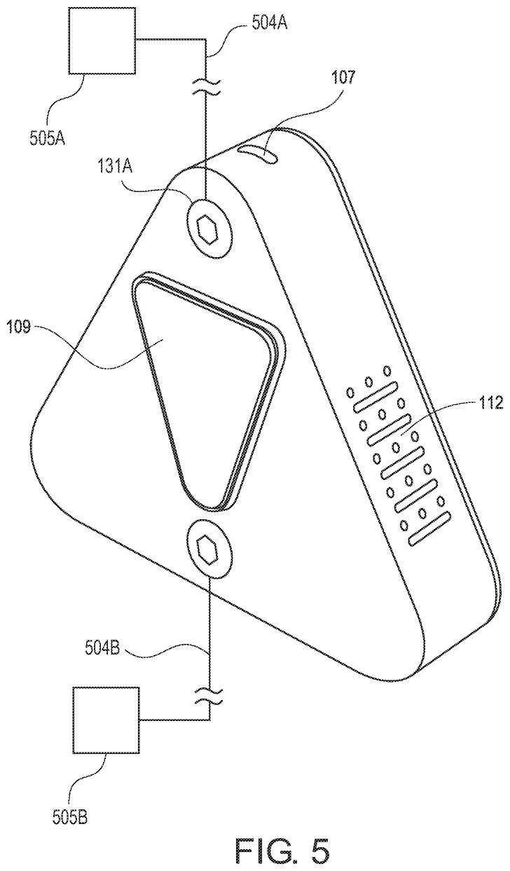

FIG. 5 illustrates the treatment device with two electrode pads 505A and 505B attached thereto. With these electrode pads, either TENS or EMS can be applied to a user as described above. The electrode pads connect to the treatment device via wires 504A and 504B. The wires connect to female connectors 131A, 131B, via mating male connectors that fit into the female connectors. The male and female connectors may be magnetic to promote a solid connection, but a snap fit connection may also be used.

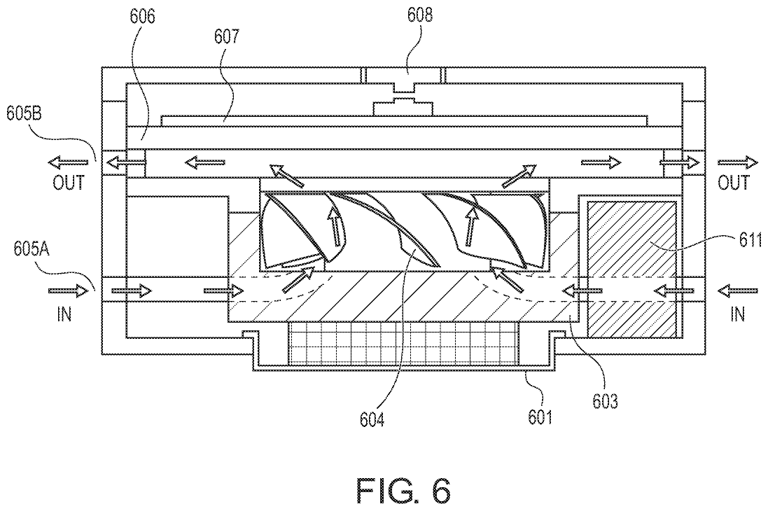

FIG. 6 illustrates a cross-sectional view of the interior components of the treatment device. An active temperature surface 601 is disposed on the bottom-most side and conducts heat or cold the user's skin. A conductive element 603 is made from metal or another conductive material and conducts electricity therethrough when electricity is supplied from the battery. A fan 604 is included to expel heat from the interior of the device. The fan is activated when the heating mode is activated to prevent the interior components from overheating. Vent passages 605A and 605B are present to provide an inlet and an outlet respectively for fan-blown air. Note that in FIG. 6, the vent passages 606A overlap some other components and are thus partially obscured in the cross-sectional view. Broken lines are used where the vent passages overlap other components. Insulation material 606 is included above the vent passages. A circuit board 607 may include the processor, memory, storage, and other microelectronic components thereon. Operation buttons 608 (only one is shown) interface with the circuit board. Finally, the battery 611 of course provides the electrical energy for the aforementioned components.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “has,” “have,” “having,” “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The explicit description of the present invention has been presented for purposes of illustration and description, but is not intended to be exhaustive or limited to embodiments of the invention in the form explicitly disclosed. Many modifications and variations will be apparent to those of ordinary skill in the art without departing from the scope and spirit of embodiments of the invention. The embodiment was chosen and described in order to best explain the principles of embodiments of the invention and the practical application, and to enable others of ordinary skill in the art to understand embodiments of the invention for various embodiments with various modifications as are suited to the particular use contemplated.

Although specific embodiments have been illustrated and described herein, those of ordinary skill in the art appreciate that any arrangement which is calculated to achieve the same purpose may be substituted for the specific embodiments shown and that embodiments of the invention have other applications in other environments. This application is intended to cover any adaptations or variations of the present invention. The following claims are in no way intended to limit the scope of embodiments of the invention to the specific embodiments described herein.

What is claimed is:

1. A handheld treatment device comprising:
a heater configured to generate heat and apply the heat to a user of the handheld treatment device;
a massager configured to vibrate and apply the vibration to the user;
an electrical energy generator configured to generate electrical energy and apply the electrical energy to the user of the handheld treatment device; and
a control interface configured to activate one or more of the heater, the massager, and the electrical energy generator.

2. The handheld treatment device according to claim 1, further comprising:
a thermoelectric cooler configured to generate a cooling effect,
wherein the control interface is further configured to activate the thermoelectric cooler.

3. The handheld treatment device according to claim 1, further comprising:
a battery power source housed inside the handheld treatment device that is configured to power the handheld treatment device.

4. The handheld treatment device according to claim 1, further comprising:
a metal plate disposed on a side of the handheld treatment device, wherein
the heater transfers the heat to the user via the metal plate.

5. The handheld treatment device according to claim 4, wherein the metal plate is curved.

6. The handheld treatment device according to claim 1, wherein the control interface further comprises a first intensity control button and a second intensity control button,
wherein activation of the first intensity control button increases a temperature of the heater, a frequency or amplitude of the vibration, or an energy amount of the electrical energy, and
activation of the second intensity control button decreases the temperature of the heater, the frequency or amplitude of the vibration, or the energy amount of the electrical energy.

7. The handheld treatment device according to claim 1, wherein the electrical energy generator is configured to generate EMS or TENS.

8. The handheld treatment device according to claim 1, further comprising a data port or wireless communication circuitry configured to communicate with an external electronic device.

9. The handheld treatment device according to claim 1, further comprising a plurality of patch connectors, and an electrode pad connected to each patch connector, the handheld treatment device being configured to apply the electrical energy to the user via the electrode pads.

10. The handheld treatment device according to claim 9, wherein a medicinal substance is disposed on the electrode pads.

11. The handheld treatment device according to claim 1 wherein a processor disposed within the handheld treatment device is configured to control the handheld treatment device to apply, at the same time:
heat from the heater;
vibration from the massager, and
electrical energy from the electrical energy generator.

12. The handheld treatment device according to claim 1, further comprising a fan disposed inside the handheld treatment device that is configured to expel air to an exterior environment.
